(19) United States
(12) Patent Application Publication
(10) Pub. No.: US 2012/0253007 A1
(43) Pub. Date: Oct. 4, 2012

(54) Synthesis of Cyclosporin H

(75) Inventors: Craig Whitaker, Annapolis, MD (US); Stephanie Caspe, Pensacola, FL (US)

(73) Assignee: The Government of the United States of America, as represented by the Secretary of the Navy, Washington, DC (US)

(21) Appl. No.: 13/355,673

(22) Filed: Jan. 23, 2012

Related U.S. Application Data
(60) Provisional application No. 61/468,316, filed on Mar. 28, 2011.

(51) Int. Cl. C07K 1/113

(52) U.S. Cl. 530/321

(57) ABSTRACT

A method of preparing purified cyclosporin H includes dissolving cyclosporin A in a first organic solvent and heating the first organic solvent in the presence of an acid catalyst; adding a base to the first organic solvent; recrystallizing cyclosporin H in a second solvent; and purifying the recrystallized cyclosporin H via chromatography to obtain purified cyclosporin H, while excluding recrystallizing the cyclosporin H in the presence of ether. In a further aspect, the method includes, after adding the base and before the recrystallizing, mixing the first organic solvent with a solvent more polar than the first organic solvent, separating the first organic solvent, and drying the first organic solvent. Cyclosporin H prepared as described herein was found to be biologically active, unlike that prepared using a previously-described method.

### CROSS-REFERENCE TO RELATED APPLICATIONS

This Application claims the benefit of U.S. Provisional Application No. 61/486,316 filed on Mar. 28, 2011, the entirety of which is incorporated herein by reference.

### BACKGROUND

A need exists for a simple and efficient means for the synthesis and purification of cyclosporin H.

### BRIEF SUMMARY

In one aspect, a method for preparing cyclosporin H includes dissolving cyclosporin A in a first organic solvent and heating the first organic solvent in the presence of an acid catalyst; adding a base to the first organic solvent; recrystallizing cyclosporin H in a second solvent; and purifying the recrystallized cyclosporin H via chromatography to obtain purified cyclosporin H, wherein the entire method excludes recrystallizing cyclosporin H in the presence of diethyl ether.

In a further aspect, the method includes, after adding the base and before the recrystallizing, mixing the first organic solvent with a solvent more polar than the first organic solvent, separating the first organic solvent, and drying the first organic solvent.

### BRIEF DESCRIPTION OF THE DRAWING

FIG. 1 is illustrates the respective structures of cyclosporin A and cyclosporin H.

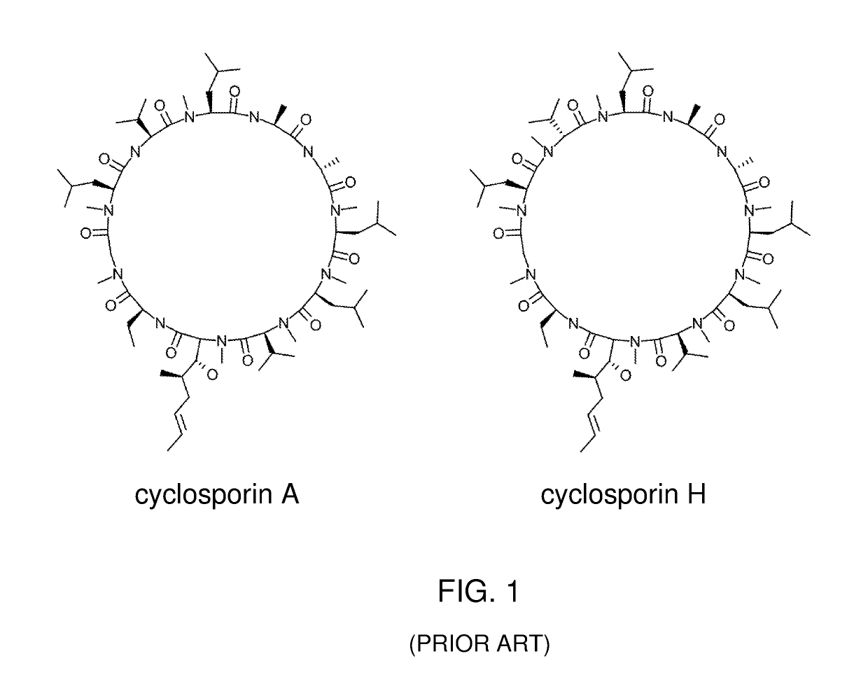

### DETAILED DESCRIPTION

### Definitions

Before describing the present invention in detail, it is to be understood that the terminology used in the specification is for the purpose of describing particular embodiments, and is not necessarily intended to be limiting. Although many methods, structures and materials similar, modified, or equivalent to those described herein can be used in the practice of the present invention without undue experimentation, the preferred methods, structures and materials are described herein. In describing and claiming the present invention, the following terminology will be used in accordance with the definitions set out below.

As used in this specification and the appended claims, the singular forms “a”, “an,” and “the” do not preclude plural referents, unless the content clearly dictates otherwise.

As used herein, the term “and/or” includes any and all combinations of one or more of the associated listed items.

### Description

Cyclosporin H is a synthetic specific competitive antagonist of formyl peptides on the formyl peptide receptor (FPR) on leukocytes.

The conversion of cyclosporin A to cyclosporin H was accomplished by the controlled heating of cyclosporin A in dioxane (or other polar organic solvent) in the presence of the acid catalyst, methanesulfonic acid (or other acid catalyst). By using a combination of heating at elevated temperature using a reflux condenser then allowing the reaction to continue to react at room temperature over the course of several days cyclosporin H was synthesized in increased yields.

The purification of cyclosporin H from cyclosporin A and from by-products of the reaction proved to be difficult. Analysis of the crude reaction mixture showed the conversion of cyclosporin A to cyclosporin H produces a significant number of side products. One document, Jegorov et al., Collect. Czech. Chem. Commun., 65, 1317-1328 (2000), describes a process consisting of heating cyclosporin A with acid catalyst for one hour then making the solution basic, extraction with organic solvent, followed by column chromatography with a methanol gradient up to 5% in dichloromethane using silica gel then multiple recrystallizations using diethyl ether. However the present inventors have found that using the method described therein resulted in a product that was not active in the studies involving leukocyte chemotaxis induced by the bacterial product f-MLP. Moreover, the material synthesized using that procedure has a broad melting point which is much lower than the pure cyclosporin H and furthermore, when analyzed by thin layer chromatography (TLC) using silica gel, produced a spot with mid-Rf value which was different then pure cyclosporin H. The TLC spot had the same Rf value as cyclosporin H but was highly fluorescent using light at 254 nm and when stained with iodine the reaction was permanent. Such properties of material made using the methodology of Jegorov et al, do not comport with those found in commercial samples of cyclosporin H. In contrast, cyclosporin H prepared as described herein was functionally active in inhibiting leukocyte migration stimulated by the standard formyl peptide bacterial product N-formyl-methionyl-leucyl-phenylalanine (f-MLP).

Removal of the unwanted by-products, particularly prior to chromatography, was found to be essential to the isolation of a purified cyclosporin H in high yield and having the desired biological activity, as determined by testing using a leukocyte chemotaxis assay. It was also discovered that the recrystallization of the synthesized material via diethyl ether (as taught by Jegorov et al.) undesirably increased the formation of one or more unwanted compounds and furthermore that the chromatographic method used was unable to remove the unwanted by-products. Furthermore, it was found that excess water in the reaction mixture after evaporation caused difficulty with column chromatography, therefore it is desirable to include one or more drying steps to remove water. Thus, a novel purification route to cyclosporin H was developed.

In a generalized exemplary synthesis, cyclosporin A is placed in dioxane and methanesulfonic acid is added. The reaction mixture is heated for one hour to reflux, then allowed to cool to room temperature and stirred under nitrogen for several days. The reaction mixture is brought to pH 9 using 10 molar NaOH. The mixture is then preferably extracted with dichloromethane and the organic layer dried with MgSO₄ followed by gravity filtration. The solvent is then evaporated to yield a white solid. Preferably, the material is then recrystallized without using diethyl ether, for example from acetone. The material is then purified using column chromatography with silica gel in combination of ethyl acetate:acetone mixtures. Impurities can be removed in this fashion, so that no UV active impurity are found in the final product. The purified cyclosporin H was found to be active in the studies involving leukocyte chemotaxis induced by the bacterial product f-MLP.

Other solvents familiar to one of ordinary skill in the art could be substituted for those described above. For example, in place of dioxane, another slightly polar organic solvent could be used to dissolve cyclosporin A. Exemplary solvents, presented in order of increasing dipole moment, include pentane, cyclopentane, hexane, cyclohexane, benzene, toluene, 1,4-dioxane, chloroform, diethyl ether, dichloromethane (DCM), tetrahydrofuran (THF), ethyl acetate, acetone, dimethylformamide (DMF), acetonitrile, dimethyl sulfoxide (DMSO), formic acid, n-butanol, isopropanol (IPA), n-propanol, ethanol, methanol, acetic acid, and water.

It is possible to use a combination of heating at elevated temperature (for example, using a reflux condenser) followed by allowing the reaction to continue to react at room temperature, thereby generating the cyclosporin H in increased yields.

Optimization of the type and quantity of acid catalyst used in the conversion of cyclosporin A to cyclosporin H can result in a high yield with fewer side reactions. It is noted that acid catalyzed degradation of the cyclosporin A to cyclosporin H causes a racemization of the starting cyclosporin A. In addition to methanesulfonic acid, other acid catalysts could be used including toluenesulfonic acid and polymeric sulfonic acids such as DOWEX resins and/or NAFION.

Analysis of a crude reaction mixture via thin layer chromatography using silica gel and alumina plates showed that the conversion of cyclosporin A to cyclosporin H produces a significant number of side products which have similar Rf values. One or more of the impurities, cyclosporin A, and cyclosporin H all have melting points which range from 120-170° C. Removal of the unwanted by-products prior to purification via chromatography (e.g., column chromatography) is critical to the isolation of cyclosporin H in high yield and having the desired biological activity.

The purification of cyclosporin H using silica gel or alumina preparatory plates produces high purity material. It is also possible to use a column for purification. Identification of the cyclosporin H is accomplished using an iodine staining method due to the lack of UV activity of the product. A variety of different solvent mixtures can be used for chromatographic purification, for example ethyl acetate:acetone or a methylene chloride/methanol gradient. Impurities can be removed by using the correct combination of solvent mixture in the appropriate order. Using a combination of polar and non-polar solvents with the silica gel and/or alumina stationary phases allows for isolation of cyclosporin H with a high yield and with high purity for use in anti-inflammatory applications.

The described purification of cyclosporin H involves the initial recrystallization of the crude synthetic material from organic solvents to remove the impurities produced in the reaction from cyclosporin A. One of ordinary skill in the art could employ multiple recrystallizations and variety of organic solvents can systematically purify the desired cyclosporin H without increasing the quantity of undesired by-products.

### EXAMPLES

### Example 1

In example 1, 20 ml of dioxane and 0.5018 g of cyclosporin A were mixed together and 0.075 ml methanesulfonic acid was added. A reflux condenser was added and the solution was refluxed for 1 hour. The pH of the solution was increased to 9 using 10 M NaOH. The solution was extracted with dichloromethane and the organic layer was evaporated to dryness on a rotator evaporator. The crude material was recrystallized from acetone to separate the unreacted cyclosporin A from the desired cyclosporin H product. The acetone layer was evaporated to dryness and the solid purified using silica gel preparatory plates with an acetone/dichloromethane mixture. Approximately, 9 mg of cyclosporin H was isolated with a melting point 150-160° C.

### Example 2

In example 2, 100 ml of dioxane and 5.0018 g of cyclosporin A were mixed together and 0.75 ml methanesulfonic acid was added. A reflux condenser was added and the solution was refluxed for 1 hour. The solution was allowed to stir at room temperature 1 hour. The pH of the solution was increased to 9 using 10 M NaOH. The solution was extracted with dichloromethane and the organic layer was evaporated to dryness on a rotator evaporator. The product was purified by column chromatography on silica gel with a mixture of dichloromethane/acetone/methanol. Approximately, 10 mg of product was isolated with a melting point 120-140° C.

### Example 3

In example 3, 20 ml of dioxane and 1.0012 g of cyclosporin A were mixed together and 0.15 ml methanesulfonic acid was added. A reflux condenser was added and the solution was refluxed for 1 hour. The solution was allowed to stir at room temperature 1 hour. The pH of the solution was increased to 9 using 6 M NaOH. The solution was extracted with dichloromethane and the organic layer was dried using anhydrous sodium sulfate. The dichloromethane layer was evaporated to dryness on a rotator evaporator. The crystals were purified via column chromatography using a dichloromethane/acetone mixture. Approximately, 10 mg of product was isolated with a melting point 130-140° C.

### Example 4

In example 4, 100 ml of dioxane and 5.009 g of cyclosporin A were mixed together and 0.075 ml methanesulfonic acid was added. A reflux condenser was added and the solution was refluxed for 1 hour. The solution was allowed to stir at room temperature for 4 days under a nitrogen atmosphere. The pH of the solution was increased to 9 using 10 M NaOH and dichloromethane was added. The solution was placed in the refrigerator for 2 days. The dichloromethane layer was isolated by extraction and the organic layer was evaporated to dryness on a rotator evaporator. The crude material was recrystallized from acetone to separate the unreacted cyclosporin A from the desired cyclosporin H product. The acetone layer was evaporated to dryness and the solid purified via column chromatography on alumina with an acetone/dichloromethane mixture. Approximately, 21 mg of cyclosporin H was isolated with a melting point 150-160° C.

Commercially purchased cyclosporin H and samples of synthesized Cyclosporin H from the above examples were tested for the ability to inhibit leukocyte migration (chemotaxis) stimulated by the standard formyl peptide bacterial product f-MLP at 10⁻⁸M. The synthesized sample #1 and sample #4 completely blocked leukocyte migration induced by f-MLP as did the commercially-purchased cyclosporin H, all at 10⁻⁵M. On the other hand, sample #2 and sample #3 of synthetic cyclosporin H at the same concentration showed partial inhibition (40-50%) of leukocyte migration induced by 10⁻⁸M f-MLP. These results indicate that methods used to prepare and purify sample #1 and sample #4 are superior to procedures used for sample #2 and sample #3.

Based on these results, it is believed that cyclosporin H prepared as described herein may be useful clinically as a potent anti-inflammatory drug. Synthesizing cyclosporin H using these new methods will allow large scale preparation of the anti-inflammatory drug at a low cost. By controlling the heating conditions, the time associated with the heating process, the choice and quantity of acid catalyst, and the type of mobile and stationary phases for column chromatography the yield of the reaction can be optimized.

All publications mentioned herein are hereby incorporated by reference for the purpose of disclosing and describing the particular materials and methodologies for which the reference was cited.

Although the present invention has been described in connection with preferred embodiments thereof, it will be appreciated by those skilled in the art that additions, deletions, modifications, and substitutions not specifically described may be made without departing from the spirit and scope of the invention. Terminology used herein should not be construed as being “means-plus-function” language unless the term “means” is expressly used in association therewith.

What is claimed is:

1. A method of preparing purified cyclosporin H, the method comprising:
(a) dissolving cyclosporin A in a first organic solvent and heating the first organic solvent in the presence of an acid catalyst;
(b) adding a base to the first organic solvent;
(c) recrystallizing cyclosporin H in a second solvent; and
(e) purifying the recrystallized cyclosporin H via chromatography to obtain purified Cyclosporin H,
wherein the method excludes recrystallizing cyclosporin H in the presence of diethyl ether.

2. The method of claim 1, wherein said first organic solvent is dioxane.

3. The method of claim 1, wherein said acid catalyst is methanesulfonic acid.

4. The method of claim 1, wherein said heating comprises reflux.

5. The method of claim 1, wherein said column chromatography is on a silica gel column.

6. The method of claim 1, wherein said recrystallizing comprises recrystallizing from acetone.

7. The method of claim 1, wherein said recrystallizing is performed at least twice.

8. The method of claim 7, wherein said recrystallizing at least twice uses an additional solvent in addition to said second solvent.

9. The method of claim 1, wherein said heating is followed by allowing continued reaction at room temperature.

10. The method of claim 1, wherein said chromatography employs a silica or alumina solid phase.

11. The method of claim 1, further comprising, after adding said base and before said recrystallizing:
mixing said first organic solvent with a solvent more polar than the first organic solvent;
separating the first organic solvent; and
drying the first organic solvent.

12. The method of claim 11, wherein said solvent more polar than the first organic solvent is dichloromethane.

13. The method of claim 11, wherein said drying comprises rotary evaporation.

14. The method of claim 11, wherein said drying comprises drying with MgSO₄ followed by gravity filtration, followed by evaporation.

15. A method of preparing purified cyclosporin H, the method comprising:
(a) dissolving cyclosporin A in a first organic solvent and heating the first organic solvent in the presence of an acid catalyst;
(b) adding a base to the first organic solvent;
(c) mixing said first organic solvent with a solvent more polar than the first organic solvent;
(d) separating the first organic solvent;
(e) drying the first organic solvent;
(f) recrystallizing cyclosporin H in a second solvent; and
(g) purifying the recrystallized cyclosporin H via chromatography to obtain purified cyclosporin H,
wherein the method excludes recrystallizing cyclosporin H in the presence of diethyl ether.

16. The method of claim 15, wherein said first organic solvent is dioxane, said solvent more polar than the first organic solvent is dichloromethane, and said second solvent is acetone.
